(19) United States
(12) Patent Application Publication
(10) Pub. No.: US 2005/0209193 A1
(43) Pub. Date: Sep. 22, 2005

(54) Method for enhanced photodynamic therapy

(76) Inventor: Gregory S. Keller, Santa Barbara, CA (US)

Correspondence Address: PERKINS COIE LLP, POST OFFICE BOX 1208, SEATTLE, WA, 98111-1208, US

(21) Appl. No.: 11/005,879

(22) Filed: Dec. 6, 2004

Related U.S. Application Data
(60) Provisional application No. 60/527,393, filed on Dec. 5, 2003.

(51) Int. Cl. A61N 1/30; A61K 31/69; A61K 31/555; A61K 31/409; A61K 31/195

(52) U.S. Cl. 514/64; 514/185; 514/410; 514/561; 604/20

(57) ABSTRACT

A method for treating a dermatopathological skin region or region of skin free of dermatopathology. In one embodiment of the invention, a method is disclosed wherein an amount of at least one photodynamic agent is applied to the skin region and photodynamic therapy (PDT) and radio frequency (RF) are applied. The applied photodynamic agent is allowed to penetrate into the skin; and the region of skin is exposed to light energy so as to generate a biologically active agent in the skin. The method includes use of RF current either prior to, simultaneous with or post PDT. The present invention is the first to utilize the benefits of PDT, IPL, and RF simultaneously. In addition, it is the first to contemplate the use of PDL for the rejuvenation of healthy skin.

### APPLICATION HISTORY

This application is a continuation of U.S. Provisional Application No. 60/527,393, filed on Dec. 5, 2003, and which is incorporated by reference as if fully disclosed herein.

### FIELD OF THE INVENTION

The present invention relates to the fields of dermatology, photodynamic therapy, and skin rejuvenation.

### BACKGROUND

The process of aging is accompanied by a variety of changes to the look and texture of the skin. Some of these changes are the result of intrinsic or chronological aging, while others are the result extrinsic aging. Intrinsic aging is the natural result of the passage of time. The skin becomes thinner and loses much of its elasticity, and the boundary between the dermis (inner layer) and epidermis (outer layer) is flattened. The dermis begins to atrophy, and the number of blood vessels in the dermis decreases. Collagen and elastin levels decrease, and fat padding begins to disappear. The loss of underlying support by fat padding and connective tissue leads to sagging.

Extrinsic aging, on the other hand, is caused by external forces such as ultraviolet radiation. Extrinsic aging is accompanied by thickening of the epidermis. Collagen and elastin levels increase, but they become disorganized. During the early stages of extrinsic aging, elastic fibers decrease in a process called elastosis. This can be followed by the development of leaky ectatic vessels, telangiectases (dilation of previously existing small or terminal vessels), actinic keratoses (solar keratoses of the horny layer of the skin), and eventually even skin cancer.

Skin cancer is classified as benign, pre-malignant, or malignant. Examples of benign skin cancers include seborrheic keratosis, pyogenic granuloma, hemangiomas, senile angioma, dermatofirbroma, keratinous cysts, keratoacanthoma, pseudolymphoma, and hypertrophic keloids. Pre-malignant skin cancers include actinic keratosis, actinic cheilitis, arsenical keratosis, thermal keratosis, Bowen's disease, leukoplakia, and dysplastic melanocytic nevi. Malignant skin cancers include basal cell carcinoma, squamous cell carcinoma, malignant melanoma, Kaposi's sarcoma, and mycosis fungoides. Due to the vast array of tumors, several treatments have been proposed and implemented. Some with great success and others with no change in the five year survival rate of the affected individuals. Some treatments were extended into the area of rejuvenation.

In the early 1990's, dermatologists began using intense pulsed light (IPL) to rejuvenate aging skin and this practice continues with moderate success today. IPL involves the application of flashing pulses of bright light to the skin. Light energy passes through the epidermis without damaging the underlying dermis. In this application the light energy treatment acts to reduce redness, hyperpigmentation, and brown spots. IPL was eventually combined with radiofrequency energy (RF), which acts in a synergistic manner with intense pulsed light to increase its effects. In addition to reducing redness, hyperpigmentation, and brown spots, IPL/RF therapy reduces wrinkles and scars and improves pore size, texture, and skin laxity.

Another light-based treatment is Photodynamic therapy (PDT), which was developed as a treatment for tumor cells, both malignant and benign. Certain tumors and tumor cells are known to selectively absorb certain dyes to a much greater extent than surrounding tissue. In PDT, tumor cells are pre-sensitized by dye tagging, then destroyed by irradiation with light of an appropriate wavelength or waveband corresponding to an absorbing wavelength or waveband of the dye. PDT has been used to treat a wide variety of malignant tumors, as well as other diseases such as psoriasis and papillomatosis. The theoretical basis behind PDT is that light energy absorbed by dye molecules is transferred to dissolved oxygen, producing a highly reactive species that kills cancer cells and destroys tumor vasculature.

The vast majority of optical power delivered to tissue in PDT eventually degrades to heat. This heat load may augment the treatment due to improved chemical reaction rates at higher temperatures. However, cells exposed to an environment where the temperature is approximately 43° C. and higher do not remain viable and it is important that surrounding healthy tissue be kept below this temperature.

The desired increased efficacy of PDT at high temperatures led to the combining of PDT with hyperthermia by Henderson et al. (“Interaction of Photodynamic Therapy and Hyperthermia: Tumor Response and Cell Survival after Treatment of Mice in Vivo,” Cancer Research 45:6071 (December 1985)). Hyperthermia is a method of treating cancer wherein the target tumor is heated to 43°-45° C. using radio frequency energy, while the surrounding healthy tissue is maintained below 43° C. A relationship exists between the time which tissue is exposed to light beam energy and the size of the surrounding zone of thermal damage caused by that light beam. This zone of thermal damage can be minimized by the use of short “bursts” or “pulses” of laser light.

In general, PDT treatment is limited by the need to deliver light internally. In addition, the depth of penetration is limited by the optical properties of the tumor tissue, generally to a depth of 2-5 mm. In certain situations, fiber optics can be inserted directly into the tumor to deliver light. However, this is often impossible for technical reasons such as tumor location and size.

A variety of improvements have been made to the general method of PDT-mediated cancer therapy. Dougherty (U.S. Pat. No. 5,257,970) teaches the combination of PDT and RF-mediated hyperthermia to treat tumors. In Dougherty, an activator component is encapsulated in a heat-sensitive liposome. After the liposome is injected, it is melted at 41°-45° C. using a heat-producing system. Upon melting, the activator component transfers energy to a previously injected photosensitizer such as porfimir sodium. This process leads to tumor eradication in at least half the treated animals. However, this treatment has certain drawbacks, certain of which are secondary to the invasive nature of the treatment.

Chen (U.S. Pat. No. 5,445,608) describes the use of an implantable light source for use in PDT at an internal treatment site. Chen envisions the use of either multiple lights or a single light capable of emitting a variety of wavelengths. Again, this treatment is invasive and technically complicated.

Meserol (U.S. Pat. No. 5,489,279) teaches a method of applying PDT to a skin lesion wherein the skin lesion has been pre-treated with a hydrogel. This hydrogel hydrates the stratum corneum, which enhances its optical and chemical transparency or transmissiveness. In this manner, the hydrogel acts as a coupling agent between the continuous light source and the photopharmaceutical. Hydrogel also creates an unwanted dispersion of light energy.

Mager (U.S. Pat. No. 5,944,748) describes a PDT device for applying light in a pattern that corresponds to the lesion to be treated. Although this ensures that light is applied only to the lesion area, and not to surrounding healthy tissue, the treatment has not gained popularity. Similarly, U.S. Pat. Nos. 5,422,093, 5,234,940, 5,079,262, and 5,955,490 to Kennedy et al., describe the treatment of rapidly growing skin cells by application of aminolevulinic acid (ALA) onto skin lesions and exposing the lesions to light.

Due to the many drawbacks of existing PDT treatments, a method that is less destructive as well as more focused is needed. Further, although the use of PDT for tumor therapy is well established, it has heretofore not been used for rejuvenation of healthy skin. The present invention contemplates the use of PDT, RF, and IPL to reduce the signs of both intrinsic and extrinsic aging on skin as well as in the treatment of tumors.

### SUMMARY OF THE INVENTION

The present invention provides a method of skin treatment including skin rejuvenation using a photosensitizing agent, or an agent that becomes a photosensitizing agent after being applied to the skin, with photodynamic therapy (PDT). In one embodiment, PDT is performed in conjunction with hyperthermia created by surface application of a radio frequency (RF) current. In another embodiment, performed using intense pulsed light (IPL) as opposed to continuous wave (CW) light to minimize the deleterious effects of constant exposure to a light source.

In another embodiment of the invention, an area of the skin is treated with a photosensitizing agent which penetrates the skin, such as porfimir sodium. RF is then applied so as to elevate the normal skin temperature. Once the skin area temperature has been so elevated, the skin area is exposed to light energy.

In another embodiment of the invention, an area of the skin is treated with an agent that does not become a photosensitizing agent until it has penetrated the skin. For example, ALA converts to a photolabile compound after exposure to light. As used herein those compounds that are active prior to penetration of and interaction with the skin are referred to photolabile compounds. Those compounds that are active after penetration of and interaction with skin are referred to as precursor PLCs or pPLCs. Precursor PLCs and PLC are referred to collectively herein as PDT agents.

RF is then applied so as to elevate the normal skin temperature. Once the skin area temperature has been so elevated, the skin area is exposed to light energy. The light energy acts to activate the PDT agent. When the PDT agent is exposed to a specific wavelength of light energy, it produces a form of oxygen that kills nearby cells.

PDT agent formulations may be used for separate embodiments of the invention and may be selected from the group consisting PDT agent plus antioxidants such as β-carotene, Vitamin C, Vitamin E, glycolic acid or moisturizer.

In separate embodiments of the invention, the light applied may be pulsed light energy or continuous wave light energy. The RF current may also be pulsed.

Embodiments of the invention may use light energy produced by an arc lamp, which is known in the art. Further, laser, filament lamp or light emitting diodes may be used.

Embodiments of the invention may be used for skin rejuvenation, acne treatment, skin bleaching, pigmented and vascular lesions, acne treatment, pre-cancerous and cancerous lesions as well as benign tumors and other general dermatopathological conditions. Further, treatment of non-pathological conditions, such as un-wanted hair, are also contemplated as aspects of the invention.

### DESCRIPTION OF SELECTED EMBODIMENTS

All references cited in this application are incorporated by reference as if fully disclosed herein.

5-aminolevulinic acid, also called δ-aminolevulinic acid, 5-amino-4-oxopentanoic acid and δ-amino-γ-keto-valeric acid, is a naturally occurring amino acid used as a photosensitizer in photodynamic therapy (PDT) of cancer and pre-cancererous conditions such as actinic keratosis. PDT is a treatment that uses a PDT agent and a particular type of light energy to activate a PDT agent. The PDT agent is a compound that generates a biologically active agent upon exposure to light energy. When the PDT agent is exposed to a specific wavelength of light energy, it produces a form of oxygen that kills nearby cells. For example, ALA is applied to the skin surface to be treated in one or two applications of a 20% by weight (w/w) formulation and then followed by a exposure to light energy.

PDT agents can are compounds that are photosensitive or photolabile prior to or upon penetration of the skin. The amounts of various PDT agents to be used are well known in the art. Hematoporphyrin derivatives (HpD) are the most commonly used photosensitizers for PDT which are photosensitive or photolabile prior to penetration of the skin. HpD exists as a complex combination of porphyrin monomers and oligomers derived from hematoporphyrin. HpD is produced by reacting hematoporphyrin with acetic and sulphuric acids, followed by an alkali work up. A purified fraction of HpD, porfimir sodium, is commonly used in PDT. Fourteen to eighteen hours after application of a PDT agent, the patient returns to the caregiver and the skin surface previously treated with the PDT agent is exposed to light energy. An example of a pPLC that is not activated, or photolabile, until it has penetrated the skin is ALA. ALA itself is not a photosensitzer but is a key metabolic precursor in the biosynthesis of the body's own naturally-occuring, iron-containing porphyrin, heme. Via biochemical reactions after skin application, the ALA becomes photosensitive.

An unexpected finding that radio-frequency (RF) current can be used for creating an effective and controlled hyperthermia within the skin that can be used in combination with photodynamic treatment of the skin serves as the basis for at least one embodiment of the invention disclosed herein. As used herein, RF refers energy derived from radiofrequencies within the range of about 300 kHz to 30 MHz. In one embodiment of the invention, RF is used in conjunction with PDT. A skin region that has been treated with a PDT agent is then fitted with electrodes which transmit RF to the skin region. As used herein, a skin region refers to any area of the skin that is selected for treatment. The skin region selected may have pathologically affected skin or not.

Certain PLCs, such as porfimir sodium, are active upon application to the skin. Other pPLCs, such as ALA are active after penetration of and interaction with, the skin and show increased activity upon exposure to light energy. Thus, ALA and many of the embodiments of this invention, become photolabile after the applied pPLC has penetrated into the skin. After application of the skin region with a pPLC, the skin region is exposed light energy in the visible and/or infrared range in combination with skin hyperthermia created by RF current.

The present invention thus provides a method for treating skin. In this disclosure, the term skin is used to refer to the epidermis, dermis and hypodermic regions that may be involved in selected dermatopathological conditions or non-dermatopathological conditions. Dermatopathology refers to any abnormality of the skin, including clinical dermatopathology, or those dermatopathological conditions that may be diagnosed visually, and those dermatopathological conditions which are diagnosed using histological laboratory analysis.

Dermatopathological conditions contemplated herein include, but are not limited to, acne, psoriasis, vascular and pigmented lesions, skin rejuvenation, actinic keratosis, sebaceous gland treatment. Additionally the method can be used for malignant lesions such as any of the malignant melanomas, basal cell carcinoma, squamous cell carcinoma and squamous cell carcinoma in situ. Moreover, it has been observed in at least one embodiment of the invention that the method reduced surface level bacteria. Thus, the method may be used for treatment of opportunistic skin infections such as acne and other bacterial related pathologies. Moreover, the method may be used for cosmetic treatments including, without being limited to, for example, skin bleaching, hair removal and pigmented lesion treatment.

In accordance with one method of the invention, a therapeutically effective amount of a PDT agent is applied to a skin region. Light energy and RF is then applied. Light energy, as used herein, refers to any source of light including, but not limited to, continuous wave light, pulsed light, inferred light, LED and laser, and combinations thereof. Interaction of the applied PDT agent with light energy generates a biologically active agent that stimulates skin metabolism and kills cancer cells.

Non-limiting examples of pPLCs that are converted into a photosensitizer in the skin include boronated protoporphyrin and derivatives of benzoprophyrine. Non-limiting examples of ALA derivatives include lipophilic ester derivatives, such as ALA hexyl ester, ALA benzyl ester, ALA pentyl ester or ALA methyl ester.

In one embodiment of the invention, the method includes the use pulsed light energy produced by flash lamp. The spectrum produced by the lamp may be filtered with standard UV or other light filters known in the art in order to remove the ultra violet portion of the produced light spectrum. The pulsed light energy may be produced by lamp, laser or LED. In another embodiment of the invention, a continuous wave (CW) light energy is used. A continuous light energy may be produced by an arc lamp, laser or LED.

As appreciated by those versed in the art, the type of filter to be selected depends on the region of the skin, e.g. the layer of the skin, and type of condition to be treated. For example, when the target region of the skin is the dermis (e.g. in case of skin rejuvenation treatment), a filter lens that filters out wavelengths shorter than 450 nm, which have a penetration depth less than 0.1 mm, is preferably used. In another example, treatment of thick psoriatic plaques or acne requires a penetration depth of up to 0.5 mm. Thus, also in this case, a filter that filters out wavelengths shorter than 450 nm is used. In yet another example, for treatment of epidermal pigmented lesions a filter that filters out wavelengths around 400 nm is optimal.

The PDT agent applied to the skin region may be formulated as oil, gel, ointment, paste, spray, stick, cream or any other form known in the art. Moreover, in another embodiment of the invention, the PDT agent may be combined with thickening agents, gelling agents, suspension agents, emulsifiers, dispersing agents depending on the desired characteristics of the formulation.

The PDT agent may also be combined with other agents known to be used with topical formulations. For example, the agent may be combined with antioxidants such retinoic acid, soy based isoflavones such as genestin, β-carotene, vitamin E and other topical additives such as vitamin C, glycolic acid or moisturizers.

In an embodiment of the invention, the PDT agent is formulated in a cream. The cream may include topical filter substances known in the art such as zinc oxide, titanium oxide, iron oxide, octyl methoxycinnamate, to name a few, or, in an alternative embodiment, the filter substance may be applied to the region of the skin directly. The PDT agent may be applied to a skin region in a formulation at a concentration of about 20% by weight. According to another embodiment, the agent is applied to the skin at a concentration higher than 2% by weight. Concentrations of PLC within the formulations may be within the range of 1% to 30% by weight of the formulation.

Hyperthermia of the skin is created by the RF current applied to the skin. The RF current may be applied prior to, with or after use of light energy. The RF current and application time should be adjusted to increase the skin temperature above the normal level but lower than a necrotic level; thermal necrotic levels can be encountered at and above 40° C., depending on skin quality and age, and are to be avoided. The optimal temperature increase is in the range of 5-10° C. above the normal level.

The optimal frequency of RF current is in the range 300 kHz to 30 MHz to create pure thermal effect, i.e., that effect without necrosis or other change associated with excess current applied to skin. The RF current may be applied through one or more electrodes applied to the skin surface. A conductive media such as water or gel can be used to serve as a coupling agent. Coupling agents are well known in the art. The coupling agent here is used for electrical coupling between electrodes and skin surface. Placement of electrodes, usually two, should be adjusted to create skin heating at the depth of about 1 mm and should correlate with light penetration depth. For example, set forth above, a light energy having a wavelength shorter than 450 nm has a skin penetration depth of about 0.1 mm. Thus, RF electrodes used with light energy having a wavelength shorter than 450 nm should be arranged to create a skin heating depth of about 0.1 mm, the skin penetration depth of the light energy.

Skin heating depth is a function related to at or about half the distance between electrodes. Electrical current, in this embodiment, is localized between 2 electrodes. By way of example, if the distance is about 0.2 mm (between electrodes) then the skin heating depth is about 0.1 mm.

The following examples utilize 5-aminolevulinic acid as the pPLC, but any PDT agent generally associated with PDT, as those mentioned above, can be used. The PDT agent may be applied to limited areas, such as keratoses or sun damaged skin, or it may be applied to an entire area. PDT agents may also be administered parenterally. Application amounts of PLCs are well known in the art.

### EXAMPLE 1

### Determination of Effective Treatment Time

The most effective time course for PDT/IPL/RF rejuvenation therapy was determined using a human test subject. ALA, was applied to the skin of the subject. The light used for PDT was IPL comprising a variety of wavelengths, with an intensity of 26 J. PDT/IPL/RF was applied at various time points following ALA application. At 15 minutes, PDT/IPL/RF was no more efficacious than standard IPL/RF alone. At 30 and 45 minutes, there was a minimal increase in efficacy over RF alone. At 1 and 2 hours, there was a noticeable increase in efficacy. This increase was even greater at 3 and 4 hours, with 4 hours showing the largest increase of any point in time. The increase in efficacy leveled off at hours 5 and 6.

A 4-hour treatment with ALA was required for significant efficacy of PDT/IPL/RF. A time course of this length may be accompanied by increases in erythema and other side effects. In order to minimize side effects, it was determined that a 1-hour treatment was preferred. The 1-hour treatment requires repeated administrations for maximum efficacy, but prevents skin breakdown. With its potential for increased side effects, the 4-hour treatment should be reserved for severe premalignant cases only.

### EXAMPLE 2

### Treatment of Patients with PDT/IPL/RF

Four patients were treated with a combination of PDT, IPL, and RF. ALA was applied to the patients at time 0.

Patient 1 received one PDT/RF/IPL peel treatment at full energy (26 J) 4 hours after application of ALA. Treatment resulted in significant darkening and slough of atypia and keratoses, as well as generalized skin rejuvenation and a diminution of wrinkles. The recovery period for this 4-hour treatment is approximately one week, with selective destruction of lentigines.

Patients 2, 3, and 4 received four sessions of PDT/RF/IPL photofacial treatments at full energy (26 J) 1 hour after application of ALA. After 4 sessions, each patient displayed a decrease in sun damage, rosacea, and melasma.

This data establishes that addition of PDT to IPL/RF photofacials enhances treatment efficacy for rosacea and sun damage. The addition of PDT also extends the use of the treatment to keratoses, and increases effectiveness against rhytids and cholasma.

Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. A method comprising the steps of:
applying to a region of skin a therapeutically effective amount of a PDT agent;
exposing the skin region to a source of light energy; and
applying an RF current to the skin.

2. The method of claim 1, wherein the PDT agent is a PLC.

3. The method of claim 1 wherein the PDT agent is a pPLC.

4. The method of claim 3 wherein the pPLC is ALA or an ALA derivative.

5. The method of claim 4 wherein the ALA derivative is a lipophilic ester.

6. The method of claim 5 wherein the lipophilic ester is selected from the group consisting of ALA hexyl ester, ALA benzyl ester, ALA pentyl ester, ALA methyl ester and combinations thereof.

7. The method of claim 2 wherein the. pPLC is selected from boronated protoporphyrin, and derivatives of benzoprophyrine and combinations thereof.

8. The method of claim 1 further comprising the step of applying the PDT agent in a formulation selected from the group consisting of oils, gels, ointments, pastes, sprays, sticks, creams and combinations thereof.

9. The method of claim 8 further comprising the step of applying a PDT agent formulation having PDT agent concentration of 1 to 30% by weight.

10. The method of claim 9 wherein the PDT agent is in the PDT agent formulation in a concentration of 20% by weight

11. The method of claim 9 further comprising the step of adding an antioxidant to the PDT agent formulation.

12. The method of claim 11 wherein the antioxidant is selected from the group consisting of retinoic acid, soy isoflavones, and Vitamin E and combinations thereof.

13. The method of claim 8 further comprising the step of adding topical additives to the PDT formulation.

14. The method of claim 13 wherein the topical additives are selected from the group consisting of β-carotene, Vitamin A, Vitamin C, glycoloic acid, moisturizer, and combinations thereof.

15. The method of claim 8 further comprising the step of applying a PDT formulation comprising an antioxidant and a topical additive.

16. The method of claim 15 wherein the antioxidant is selected from the group consisting of retinoic acid, soy isoflavones and Vitamin E and combinations thereof.

17. The method of claim 15 wherein the topical additives are selected from the group consisting of β-carotene, Vitamin A, Vitamin C, glycoloic acid, moisturizer, and combinations thereof.

18. The method of claim 1 wherein the source of light energy is selected from the group consisting of LED, laser light, arc lamp, filament lamp and combination thereof.

19. The method of claim 18 further comprising the step of exposing the skin region to the source of light energy in pulsed or CW and combinations thereof.

20. The method of claim 1 further comprising the step of applying the RF current to the skin region at range of 300 kHz to 30 Mhz.

21. The method of claim 20, further comprising the step applying RF current to the skin region prior to exposing the skin region to the source of light energy.

22. The method of claim 1, further comprising the step of applying to the skin the RF current while exposing the skin region to the source of light energy.

23. The method of claim 1 further comprising applying a pulsed RF current to the skin region.

24. A method comprising the steps of:
applying to a skin region a therapeutically effective amount of an ALA;
exposing the skin region to a source of light energy; and
applying an RF current to the skin.

25. The method of claim 24 further comprising the step of applying the ALA in a formulation selected from the group consisting of oils, gels, ointments, pastes, sprays, sticks, creams and combinations thereof.

26. The method of claim 25 further comprising the step of adding an antioxidant to the ALA formulation.

27. The method of claim 25 further comprising the step of adding a topical additive to the ALA formulation.

28. The method of claim 25 further comprising the step of addition of both an antioxidant and a topical additive to the ALA formulation.

29. The method of claim 25 further comprising the step of applying an ALA formulation with 1 to 30% ALA by weight in the formulation.

30. The method of claim 24 wherein the source of light energy may be selected from the group consisting of LED, laser light, arc lamp, filament lamp and combinations thereof.

31. The method of claim 24 further comprising the step of applying the source of light energy in pulsed or CW and combinations thereof.

32. The method of claim 24 further comprising the step of applying the RF current to the skin region in the range of 300 kHz to 30 MHz.

33. The method of claim 24, further comprising the step of applying the RF current prior to exposure of the skin region to the source of light energy.

34. The method of claim 24, further comprising the step of applying the RF current to the skin region while exposing the skin region to the source of light energy.

35. The method of claim 24 further comprising the step of applying a pulsed RF current to the skin region.

36. A method of skin rejuvenation comprising:
applying to a skin region a therapeutically effective amount of ALA;
exposing the skin region to pulsed light energy using four separate one hour treatments; and
applying RF current to the skin region during the four separate one hour pulsed light energy treatments.
